(19) United States
(12) Patent Application Publication
(10) Pub. No.: US 2025/0311963 A1
(43) Pub. Date: Oct. 9, 2025

(54) Systems and Methods for Predicting an Onset of a Neurological Event in a Human or Animal

(71) Applicant: Northeastern University, Boston, MA (US)

(72) Inventors: Pedram Johari, Medford, MA (US); Ali Saeizadeh, Arlington, MA (US); Tommaso Melodia, Newton, MA (US); Jorge H. Jimenez, Boston, MA (US)

(21) Appl. No.: 19/094,041

(22) Filed: Mar. 28, 2025

Related U.S. Application Data
(60) Provisional application No. 63/571,560, filed on Mar. 29, 2024.

(51) Int. Cl. A61B 5/00; A61B 5/318; A61B 5/369; G16H 20/17; G16H 20/40; G16H 50/30

(52) U.S. Cl. CPC A61B 5/4076; A61B 5/318; A61B 5/369; A61B 5/7267; A61B 5/7275; G16H 20/17; G16H 20/40; G16H 50/30; A61B 2560/0431

(57) ABSTRACT

Systems and methods for predicting an onset of a neurological event in a human or animal are disclosed. A portable system may include one or more sensing modules configured to acquire one or more physiological signals from the human or animal. The portable system may further include a processing module communicatively coupled to the sensing module and configured to analyze the one or more physiological signals acquired. Analyzing may include employing one or more metrics of the one or more physiological signals to calculate a likelihood of the onset of the neurological event and identifying a risk period of the onset of the neurological event based on the likelihood calculated. Identifying the risk period of the neurological event prior to the onset may enable patients or caregivers to undertake preventative measures or therapeutic interventions.

### RELATED APPLICATION

This application claims the benefit of U.S. Provisional Application No. 63/571,560, filed on Mar. 29, 2024. The entire teachings of the above application are incorporated herein by reference.

### GOVERNMENT SUPPORT

This invention was made with government support under Grant No. 2214013 from National Science Foundation. The government has certain rights in the invention.

### BACKGROUND

Patients that experience unpredictable adverse neurological events, e.g., epileptic seizures, may experience lower quality of life due to, as non-limiting examples, the adverse neurological events, fear or anxiety thereof, or risk of injury during the neurological events. Current technologies may reduce the possibility of occurrence of a neurological event using medication, e.g., antiepileptic medication for epilepsy, or apply a therapeutic intervention, e.g., neurostimulation, after initial onset of the neurological event to reduce the intensity thereof. However, development of systems and methods capable of predicting a neurological event prior to an onset thereof may be helpful for notifying a patient to undertake safety measures or deliver prophylactic treatment, as non-limiting examples, and may significantly improve quality of life for patients who experience adverse neurological events.

### SUMMARY

Example embodiments of the present invention may include devices, systems, software, hardware, and methods for the use of efficient deep learning algorithms for detection, prediction, identification, or characterization of neurological events.

In an example embodiment, a portable system for predicting an onset of a neurological event in a human or animal includes one or more sensing modules configured to acquire one or more physiological signals from the human or animal. The portable system further includes a processing module communicatively coupled to the one or more sensing modules and configured to analyze the one or more physiological signals acquired. Analyzing the one or more physiological signals includes employing one or more metrics of the one or more physiological signals to calculate a likelihood of the onset of the neurological event and identifying a risk period of the onset of the neurological event based on the likelihood calculated. The risk period may be a period or length of time following calculating the likelihood of the onset of the neurological event.

In some embodiments, the processing module is configured to calculate the likelihood of the onset of the neurological event by executing a machine learning algorithm. In some further embodiments, the machine learning algorithm includes a supervised learning, unsupervised learning, reinforcement learning, natural language processing, evolutionary, ensemble, or deep learning algorithm. In some still further embodiments, the deep learning algorithm includes a convolutional neural network, pruned convolutional neural network, recurrent neural network, long short-term memory network, generative adversarial network, autoencoder, deep belief network, or multilayer perceptron.

In other embodiments, the processing module includes a training module. The training module is configured to perform at least one of training the machine learning algorithm using a repository including data of a similar type to the one or more physiological signals or refining a pre-trained machine learning algorithm using patient-specific data of a similar type to the one or more physiological signals.

In some embodiments, the one or more sensing modules or the processing module is further configured to partition the signal of the one or more physiological signals into a plurality of intervals. For example, for a physiological signal acquired over a period of time, e.g., 1 minute, an interval of the at least one interval includes a segment of time length 0 seconds to 1 minutes of the physiological signal acquired, e.g., the at least one interval may include two 30 second intervals. The processing module can be further configured to employ one or more metrics of an interval of the plurality of intervals to calculate a respective likelihood of the onset of the neurological event and to identify the risk period based on the respective likelihoods calculated.

In other embodiments, the likelihood calculated is a first likelihood and the processing module is configured to employ the one or more metrics of the one or more physiological signals to calculate at least one additional likelihood. In some further embodiments, the processing module includes a voting module configured to form the prediction based on the likelihood and the at least one additional likelihood calculated.

In some embodiments, the one or more sensing modules are configured to acquire an electrocardiography, electroencephalography, temperature, heart rate, accelerometry, electromyography, or electrodermal signal.

In other embodiments, the one or more sensing modules acquire physiological signals using at least two physiological measurement modalities. The one or more sensing modules may further acquire one or more physiological signals of each of the at least two physiological measurement modalities.

In some embodiments, the sensing module and the processing module are configured to communicate via a communications path including at least one link employing a wireless communication protocol. In some further embodiments, the wireless communication protocol is a Bluetooth-based communication protocol or an ultrasound-based communication protocol.

In some embodiments, the portable system further includes a therapeutic module communicatively coupled to the processing module. The therapeutic module is configured to apply a therapeutic intervention based on the prediction of the onset of the neurological event. In some further embodiments, the therapeutic module includes a drug infusion pump or a neurostimulation device.

In some embodiments, the neurological event is an epileptic seizure, a tremor, or a migraine.

In other embodiments, the processing module is configured to analyze the one or more physiological signals within a proximity of short-range wireless communication from the sensing module. Re-stated, the processing module is configured to analyze the one or more physiological signals locally (as opposed to remotely or on a cloud server). Performing the analyzing locally may allow a patient to receive the prediction without a need for long-distance wireless communication or without a latency period.

In another example embodiment, a method for predicting an onset of a neurological event in a human or animal includes acquiring one or more physiological signals of the human or animal. The method further includes analyzing the one or more physiological signals, including employing one or more metrics of the one or more physiological signals to calculate a likelihood of the onset of the neurological event. Analyzing the one or more physiological signals further includes identifying a risk period of the onset of the neurological event based on the likelihood calculated.

In some embodiments, calculating the likelihood of the onset of the neurological event includes executing a machine learning algorithm. In some further embodiments, the method further includes training the machine learning algorithm based on a repository including data of a similar type to the one or more physiological signals. The method still further includes refining the machine learning algorithm trained using the one or more physiological signals acquired of the human or animal.

In some embodiments, the method further includes delivering a therapeutic intervention to the human or animal based on the prediction formed. For example, delivering the therapeutic intervention may include prompting the human to take medication, injecting an infusible medication, or delivering a stimulus pulse.

In other embodiments, the method further includes partitioning the signal into a plurality of intervals. Analyzing the one or more physiological signals may include employing one or more metrics of an interval of the plurality of intervals calculate a respective likelihood of the onset and identifying the risk period based on the respective likelihoods calculated.

In some embodiments, the calculating the likelihood of the onset of the neurological event includes calculating at least two likelihoods and identifying the risk period based on the likelihood calculated includes performing a voting scheme on the at least two likelihoods.

In another example embodiment, a method for predicting an onset of a neurological event in a human or animal includes employing one or more metrics of at least one physiological signal acquired from the human or animal. The method further includes identifying a risk period of the onset of the neurological event based on the likelihood calculated. The method still further includes forwarding a representation of the prediction formed to the human or animal, to a caregiver of the human or animal, or to a therapeutic module arranged to apply a therapy to the human or animal.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A schematically illustrates an example embodiment of a portable system for predicting an onset of a neurological event in a human or animal.

FIG. 1B illustrates a flowchart of an example embodiment of a method for predicting an onset of a neurological event in a human or animal.

FIG. 2 illustrates a sensing module of an example embodiment of a portable system for predicting an onset of a neurological event in a human or animal.

FIG. 3 illustrates a processing module of an example embodiment of a portable system for predicting an onset of a neurological event in a human or animal.

FIG. 4A illustrates an example embodiment of a portable system for predicting an onset of a neurological event in a human or animal including electrical leads for measuring an electroencephalography (EEG) signal or for delivering a therapeutic stimulation, with inset images showing an example electrical lead.

FIG. 4B illustrates a sensing module of a system similar to the portable system of FIG. 4A, with insets showing an external charger and a handheld device to which the portable system is communicatively coupled.

FIG. 4C illustrates a processing module of a system similar to the portable system of FIG. 4A, with insets showing example wearable embodiments of processing modules.

FIG. 5A illustrates an example embodiment of a processing module of a portable system for predicting an onset of a neurological event in a human or animal.

FIG. 5B schematically illustrates an example embodiment of a portable system for predicting an onset of a neurological event in a human or animal including a processing module communicatively coupled to a therapeutic module configured to deliver therapeutic intervention for the neurological event.

FIG. 5C schematically illustrates an example embodiment of a portable system for predicting an onset of a neurological event in a human or animal, the system including a sensing module with implantable electrical leads for measuring electroencephalography signals and a wearable processing module.

FIG. 6 illustrates an architecture of a convolutional neural network that may be implemented by an example embodiment.

FIG. 7 illustrates a voting scheme that may be implemented in an example embodiment of a portable system for predicting an onset of a neurological event in a human or animal, the system configured to acquire intracranial electroencephalography and electrocardiography signals.

FIG. 8 illustrates a plot of average sensitivity, specificity, and accuracy of epileptic seizure prediction across a cohort of subjects according to an example embodiment.

FIG. 9 illustrates a plot comparing area under the curve metrics for an example embodiment of the present invention and a previously reported epileptic seizure prediction technique.

FIG. 10 illustrates a computer network or similar digital processing environment in which embodiments may be implemented.

FIG. 11 is a diagram illustrating an example internal structure of a computer in the environment of FIG. 10.

### DETAILED DESCRIPTION

A description of example embodiments follows.

Epilepsy is a common neurological disorder disease, with about 65 million people diagnosed worldwide, and may carry a risk of premature death three times higher than that of the general population. Although most patients diagnosed with epilepsy respond well to pharmaceutical drugs for treating epilepsy, approximately one-third of epilepsy patients suffer from drug resistant epilepsy. Therefore, there may be a need for alternative epilepsy treatments beyond pharmaceutical care. In recent years neuromodulation techniques and technologies, including deep brain stimulation devices, have been developed for the treatment of epilepsy and other neurological disorders such as Parkinson's Disease (PD), migraines, Alzheimer's Disease (AD), and chronic pain, among others. Although such devices or systems and innovations in anti-seizure medication may improve patient outcomes and quality of life, existing treatment modalities may suffer from a significant shortcoming. Specifically, many existing treatment modalities may detect an occurrence of a neurological event after onset and aim to deliver a therapeutic treatment that reduces the intensity or effect of the neurological event. By contrast, developing treatment techniques capable of predicting the occurrence of a neurological event prior to onset may empower patients or caretakers thereof to undertake risk mitigation measures or to provide therapeutic interventions that may prevent or mitigate the neurological event itself.

Currently, antiepileptic medication for epilepsy may be used to reduce the possibility of a seizure occurring or reducing its intensity; neurostimulation may generally be initiated after initial onset in order to reduce its intensity. Treatment for PD, AD, migraines, and other neurological events may follow a similar philosophy, providing reactive treatment following onset as opposed to prophylactic. Furthermore, and specifically for epilepsy, the actual lack of information or warning about the seizure onset may result in poor quality of life, for example, due to fear or anxiety from patients, which limits their daily activities. The lack of warning may also result in a high risk of injuries and hospitalization of patients since they are unable to take precautions prior to the onset of the event. Therefore, a system that can predict seizure with enough time in advance to the onset of a seizure may subsequently inform the patients for peace of mind, for pre-emptive treatment, or for taking appropriate precautions and may result in a large improvement in quality of life and patient outcomes, including reduced risk of injury and reduced cost burden on healthcare systems.

Different approaches have been taken for predicting onsets of neurological events, including the use of artificial intelligent in predictive algorithms for prediction of epileptic seizures. However, current approaches may include significant shortcomings. A first shortcoming may include a use of multiple signals from the body and a use of significantly large processing power, notably in cloud processing, in order to predict a potential event. This process may imply that a system using such a method needs to be connected continuously to the internet, have a significant lag time, and as a result may be impractical for day to day use. Other techniques may rely on EEG signals from implanted electrodes or multiple patches on a head of a patient. Such systems, which may include up to 15 electrodes, may not be practical for 24 hour use in everyday life and may propagate stigmas associated with patients living with epilepsy and increase isolation thereof from society and personal relationships. Furthermore, recent systems with a reduced number of signals, typically associated with iEEG or EEG, may suffer from low accuracy and high rates of false positives and false negatives in their predictive capabilities.

As such, development of systems and means for predicting an occurrence of neurological events prior to an onset would be very beneficial for epileptic patients and general neurological patients, who may from unpredictable adverse events, such as seizures for epilepsy, severe migraines, or other neurological events. Having access to such a system or means in situ (on the body, for example, a wearable or implantable device) that can predict such adverse event by processing data locally (no need for internet connectivity) in a reliable, accurate and energy energy-efficient manner and using a minimal number of signals may be helpful for facilitating daily life and reducing risk factors for patients with such neurological disorders.

Example embodiments of the present invention may include systems and methods for predicting an onset of a neurological event, particularly performing the predicting locally. As defined herein, locally may include within a spatial proximity of a patient, e.g., on a wearable or portable device. Additionally, performing the predicting locally may not require transfer of data representative of physiological signals over great distances, for example, to a cloud server. An example of local processing may include within a short-range wireless communication range, wherein data may be transmitted locally using a communication path that includes Bluetooth® communication or another wireless transmission protocol, and processed within the short-range wireless communication range from the patient and sensors configured to detect a physiological signal of the patient.

Embodiments of the inventions described herein were tested using signals from an epileptic patient database (EPILEPSIAE dataset, data is stored in a PostgreSQL database with a relational structure), which may be used to train personalized prediction algorithms and demonstrate full predictive function for epileptic seizures. Accuracy, sensitivity, and specificity of algorithms that used iEEG, EEG and ECG signals by themselves or a combination thereof are tested at different seizure prediction time windows using data from a separate iEEG/EEG/ECG dataset.

### Systems and Devices for Detecting an Onset of a Neurological Event

In some example embodiments of a system for detecting an onset of a neurological event in a human or animal, the system may include a sensor used to acquire signals from the human body to identify, characterize, and or predict a neurological event. In general, a system including a single device capable of sensing and processing, or multiple devices that could sense and process are used to sense a signal from a part of the human body and processes such signal in order to identify, characterize, and or predict a neurological event. In some embodiments, a sensor module and processing module are housed in a single device, whereas in other embodiments, a sensing device with a sensing module and a processing device with a processing module are housed in different devices that communicate with each other. In some embodiments of the system there may be multiple devices capable of processing and multiple devices capable of sensing signals. In a particular one of the embodiments, two devices are used, one that contains a sensing module and one that contain a processing module, with the ability of sending date data between them. In another example embodiment, a single processing device with a processing module can communicate with multiple sensing devices with sensing modules.

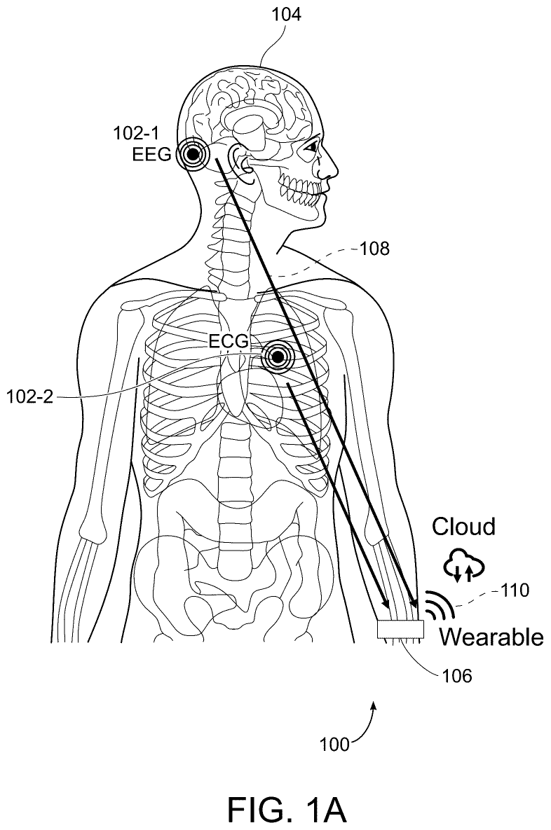

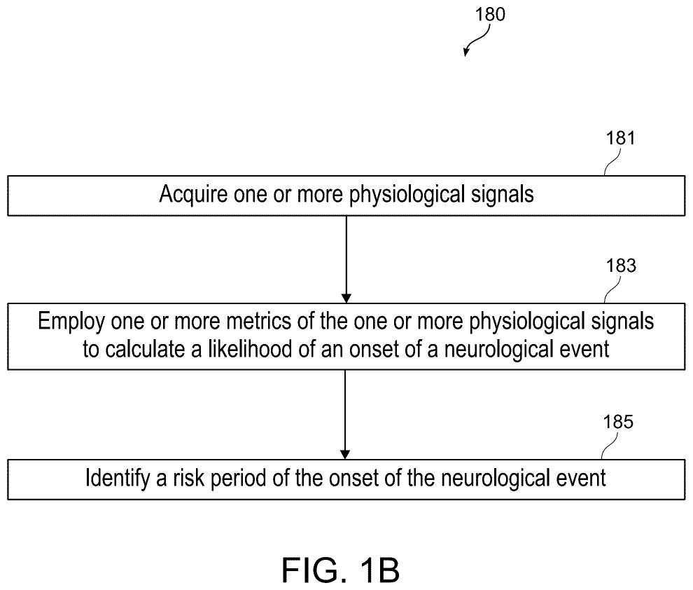

FIG. 1A schematically illustrates an example embodiment of a portable system 100 for predicting an onset of a neurological event in a human 104 or animal. The portable system 100 includes one or more sensing modules, e.g., 102-1, 102-2, configured to acquire one or more physiological signals from the human 104. A signal of the one or more physiological signals may include at least one interval. Example physiological signals may include an EEG signal 102-1 or an ECG signal 102-2. The portable system 100 further includes a processing module 106 communicatively coupled to the one or more sensing modules, e.g., 102-1, 102-2. The processing module 106 is configured to analyze the one or more physiological signals acquired. Analyzing may include employing one or more metrics of the one or more physiological signals acquired to calculate a likelihood of the onset of the neurological event and identifying a risk period of the onset of the neurological event based on the likelihood calculated. The risk period may be a period of time, e.g., a few seconds, a few minutes, an hour, or a further extended period of time, during which the onset of the neurological event is predicted to occur.

The processing module may be communicatively coupled to the one or more sensing modules, e.g., 102-1, 102-2, through communication paths, e.g., 108. The communication paths, e.g., 108, may include a wired communication path or a wireless communication path, for example, Bluetooth® or wireless fidelity (Wi-Fi). In some example embodiments, the communication path, e.g., 108, may utilize short-range wireless communication for local transmission of acquired signals. The processing module 106 may be further configured to communicate with other devices, for example, a cloud server, a therapeutic module, or a caretaker or healthcare provider. In some embodiments, the processing module 106 may include an additional communication path 110 configured for transmitting data over short or long distances.

In some embodiments, the processing module, e.g., the processing module 106, may include a calculation module configured to employ one or more metrics of the one or more physiological signals to calculate a likelihood of the onset of the neurological event. In other embodiments, the processing module may include a risk period module configured to identify a risk period of the onset of the neurological event based on the likelihood calculated, for example, by a calculation module.

Several embodiments may be used to acquired different types of signals through different sensor types, which may then be processed through a processing device or module. Signals that may be acquired using a sensor or sensing module, for example, the one or more sensing modules, e.g., 102-1, 102-2, include but are not limited to two electrical (EEG, ECG, peripheric nerve voltage/amperage/resistance, tissue resistance, nerve electrical conductivity, muscle conductivity, cardiac muscle electric signals beyond ECG, iEEG), temperature (skin temperature, implant temperature), mechanical (pressure, strain, force), optical (infrared skin optics, biometrics, facial features, oxygenation, reflectivity, refraction, conductance). In some embodiments, laminar flow within vessels may be measured for determination of vessel occlusion, for example, by a thrombus or plaque, which may be useful for pre-peripheral artery disease intervention. In other embodiments, one, multiple, or all of such types of physiological signals may be acquired through sensors, sensor modules, or sensor devices to be used by themselves or in combination when training or executing the deep learning algorithms. In a particular embodiment, a number of signals used may be limited to one or two signal types in order to be able to conduct AI-based evaluation, through a deep learning method, in situ (on the body) through an implant, wearable, or other device that may be carried by the patient itself. Signal characteristics and or metrics including, but not limited to, characteristics/metrics in the amplitude domain, time domain, frequency domain, phase domain, and waveform domain may be used by AI algorithms. In an example embodiment using ECG signals, characteristics of the QRS complex and its changes in time may be used by an AI algorithm as metrics to predict an onset of an epileptic seizure. In some embodiment the amplitude of the different peaks of the QRS complex or spacing of the different peaks in time may be used as a predictor metric. In other embodiments a change in a shape of the QRS waveform may be used. In further embodiments, changes in shape or frequency of the QRS complex may be used.

FIG. 1B illustrates a flowchart of an example embodiment of a method 180 for predicting an onset of a neurological event in a human or animal. The method includes acquiring one or more physiological signals of the human or animal 181. The method further includes employing one or more metrics of the one or more physiological signals to calculate a likelihood of the onset of the neurological event 183. The method still further includes identifying a risk period of the onset of the neurological event 185 based on the likelihood calculated.

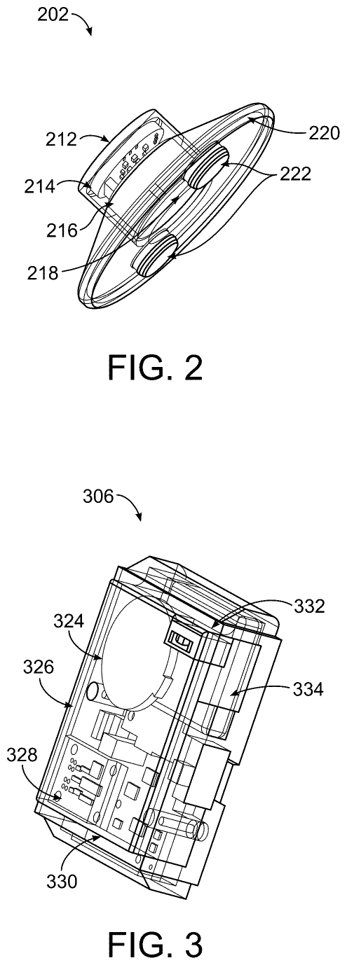

FIG. 2 illustrates a sensing module 202 of an example embodiment of a portable system for predicting an onset of a neurological event in a human or animal. The sensing module 202 includes an electronics casing 212 enclosing electronic components. The sensing module 202 further includes a communication board 214, e.g., a Bluetooth communication board, configured to communicate with a processing module, e.g., the processing module 106 described herein with reference to FIG. 1A. The sensing module 202 further includes sensing elements, e.g., electrodes 222, and a sensing board 216. The sensing board 216 may be used to convert an analog signal to a digital signal and perform signal pre-conditioning or pre-processing prior to transmitting the signal to the processing module. The sensing module 202 further includes a rechargeable battery 218, which may enable wireless operation, and an adhesive patch 220, which may ensure sufficient contact between the sensing elements and the human or animal.

In some embodiments, iEEG signals from an implantable system such as a peripheral nerve stimulation sensor or lead, brain stimulator (including deep or shallow variations), or implantable brain sensors or leads may be used to acquire signals for a sensor module or device. The signals acquired for the sensor module or device may be sent to a processing module/device in order to execute an AI-based software based on the signals acquired. In such embodiments, the physical layer short-range and long-range communication may be radiofrequency (RF)-based or ultrasound-based. Ultrasound communication channels transmitting through tissue may be considered for transmitting and receiving data between implanted and wearable devices that executes data sensing (e.g., iEEG or ECG sensing), classifications, or processing and a wearable device, or just between implanted devices themselves. A short-range (few mm) ultrasonic link may enable embodiments to perform focused propagation with lower transmission power that conserves energy for the implant. This may be a useful aspect for the implant as charging operations for an implanted device may be more complicated than that of wearable elements. In such embodiments with an implanted device, energy buffer sizes may need to be small to be easily implanted.

In other embodiments, EEG signals from head-worn sensor patches may be used to train and execute deep learning algorithms. In such embodiments, using signals of the brain from non-implanted devices, preferred signals may be acquired from EEG patches or sensor modules and devices located behind an car of a patient and transmitted to a processing unit to be used to train and execute an AI model in the processing device or module. Signals from sensors behind one or both cars (including periauricular, occipital, or mastoid regions) may be preferred for daily use, although other embodiments may include sensors in the frontal, temporal, parietal, zygomatic and other regions of the head and face.

In some example embodiments, electrical signals may be acquired from a heart of a human or animal through electrode patches placed on skin of the human or animal, although signals from implanted cardiac leads may also be used. In such embodiments, ECG signals from one or more patches, or sensor locations, may be used to train and execute deep learning algorithms. In such embodiments, a single signal from a single patch with electric sensing electrodes may be uniquely used to conduct training and execution of AI deep learning algorithms. As disclosed herein, experiments using such an embodiment, using only a single ECG signal, may allow for over 90% accuracy in predicting an onset of a neurological event with reduced sensing and processing requirements. In other example embodiments, EEG and ECG signals may be combined when training and executing AI algorithms in the processing unit. Combinatorial usage of different sensing modalities may increase accuracy of trained and executed AI models.

In some embodiments, processing and sensing modules may be combined into a single device. However, in some particular embodiments, sensing modules and processing modules are located in different devices. The sensor device may include in some embodiments the sensor itself (electric sensor, thermal sensor, optical sensor, mechanical, motions, among others) or may communicatively couple to the sensor through a wired or wireless communication path. In some further embodiments, the sensor module is in a sensor device, which may be releasably wired directly to the sensor itself. A given sensor module may also include some processing power or circuitry to filter or pre-process an acquired signal before transmitting it to a processing unit. The sensing module may be self-powered with a battery, capacitor, or other energy storage means, or it may be powered remotely by wired or wireless means. The sensing module, when located in a sensing device that is housed independently of the processing device, may also include a communication module; such a communication module may transmit data between itself and the processing device through wired or wireless means. In some embodiment, wireless means may be preferred. Example wireless means for transferring data may include data transmission through optical (infrared, visible light, other), acoustic, or electromagnetic means. In some embodiments, ultrasound waves may be used to transmit data through a body of a human or animal or air between the sensing device and the processing device.

When acoustic communication-based systems are used, an impulse-based transmission, i.e., a pulse position modulation (PPM), with a superimposed spreading code, may be used.

According to an example embodiment, data associated with or representative of decisions generated using AI algorithms may be transferred, and outcomes of the AI algorithms, e.g., DL models, may be encoded into Bapp bits (application bits) and transmitted every tapp seconds. Consequently, a minimum required bit rate Rapp for each node may be calculated as Rapp=Bapp/tapp in bits per second (bit/s). In some embodiments including concurrent communication between multiple devices (for example, a system with two ECG sensor, an EEG sensor, and a processing device), a total bit rate Rtotal may equal 4×Rapp. A time resolution of the example system is 4 s, implying tapp=4 s for all nodes of communication. In such embodiments, a simplified centralized medium access control (MAC) mechanism may be used. This centralized MAC protocol is chosen due to a given system's inherent characteristics: a fixed number of nodes and a processing device that may connect to the internet. Initially, the processing device coordinates other nodes (sensor devices) by dispatching a control message encompassing a spreading code and time-hopping frame sequence assigned to each sensor node.

The spreading code employed and time-hopping frame sequence enable multiple nodes to effectively share the channel, enabling simultaneous communication. Such a communication protocol may obviate a necessity for control messages to synchronize and mutually exclude nodes, a challenging task in ultrasonic communications due to extended and unpredictable propagation delays. A MAC protocol may proficiently support all nodes.

Using such an embodiment of a MAC protocol, each node may be allocated as follows:

$${R}_{app} = \frac{{R}_{total}}{{N}_{nodes}} = 5 kbit / s$$

Some example embodiments of a system for predicting an onset of a neurological event may transmit binary classification results generated by an AI model and may operate within a time resolution of several seconds, e.g., 4 s. For such example embodiments, the aforementioned bandwidth allocation may be more than adequate.

In a particular embodiment, wireless electromagnetic means are used to transmit data between sensing devices and processing devices, including unidirectional, bidirectional, or omnidirectional communication. Preferred wireless electromagnetic means for data transmission may include Bluetooth, Wi-Fi, medical implant communication system (MICS), cellular networks, satellite communication, microwave communication, or radio broadcast.

In other embodiments, sensor devices may include electric sensing electrodes that are located on a surface in contact with skin of a human or animal. Such sensors connect through wires to other components of a sensing module. Furthermore, such wires may be connected or disconnected at will. In some further embodiments, the sensing electrodes are located within a patch that maybe adhered temporarily or permanently to the skin of the patient. Patches may or may not be waterproof. In some embodiments, waterproof patches may be worn during contact with water (e.g., shower, bathing, submerging). In other embodiments, the waterproof patches may be releasably connected to encased electronics of the sensor unit through wires so that electronic components may be removed, but the patch may be left in place during periods of contact with water. In some further embodiments, an entire sensing device or system may be configured to be waterproof. For such a system or device components, e.g., patches, electrodes, and electronics, may remain in place during contact with water.

Patches containing or not containing electrodes may be detachable from other electronic components so that a given patch may be replaced after a given period of time (e.g., hourly, daily, weekly, monthly, yearly, or another specific period of time) during extended use of electronic components of a system, including sensing and processing modules. In some embodiments, both the patches and the electronics may be configured to be disposable, for example, disposing an entire system after a specific period of time.

In some embodiments, electronics of sensing modules, electrodes, and adhesive patch are encased in an elastomeric material. In other embodiments, the patch and electrodes include or are encased in an elastomeric-based material while the electronics are encased in a harder case. Alternative combinations of materials for construction of electronics, modules, and patches that may provide for durability of an example embodiment of a system of the present invention and for good contact between a surface of skin of a human or animal at a desired anatomical location may be used. Furthermore, electronics may include rigid or mechanically flexible circuitry boards or a combination thereof.

For some embodiments of a system for predicting an onset of a neurological event in a human or animal, optical sensors, mechanical sensors, biological sensors, temperatures sensors, chemical sensors, or other electrical sensors may be utilized in a similar way to that described for the electrodes in embodiments described herein.

According to some example embodiments, a single or multiple processing modules may be used a single or multiple processing modules or devices. In a embodiment, the single or multiple processing units may be configured to be portable such that a patient may carry the processing units with them, the processing units configured to execute an AI algorithm in situ. For example, the processing unit may be wearable or may fit in clothing of a patient. In a embodiment including portable processing units, the processing units may be worn directly on a body of a human or animal through a watch, band, harness, belt, or other source of support for the body. In other embodiments, the processing device may be configured to be carried on a person, including but not limited to pocket-sized devices (e.g., a cell phone or a tablet device).

FIG. 3 illustrates a processing module 306 of an example embodiment of a portable system for predicting an onset of a neurological event in a human or animal. The processing module 306 includes a communication module 324, which may be configured to communicate with a communication board 214 of a sensing module 202 as described herein with reference to FIG. 2. The communication module 324 may further be configured to perform signal conditioning or pre-processing. The communication module 324 further includes memory 328 that may be used for storing or buffering data, for example, physiological signals, or other inputs or outputs, e.g., metrics determined or predictions formed by the portable system. The processing module 306 further includes a rechargeable battery 334 and a light emitting diode (LED) 332 and may optionally include a touch screen 326 to enable interfacing with a patient or caretaker of the patient.

In some embodiments, a processing unit may be configured to be implantable and may be similar to, for example, a generator of neurostimulators or deep brain stimulators, among others.

According to some embodiments, at least a portion of AI processing may be conducted using a sensing device. In other embodiments, all of the AI processing may be performed in a processing unit. While in some embodiments, at least a part of or an entirety of AI processing may be performed by a non-local server using cloud-based communication and computation, particular embodiments may utilize local, in situ AI processing. In such particular embodiments, computational power consumption may be a design constraint. Computation may be performed using a processor including field programmable gate arrays (FPGAs), microprocessors, miniaturized processors, application-specific independent circuit (ASIC), or other type of miniaturized programmable hardware. According to a particular embodiment, an FPGA may be used to execute deep learning algorithms.

In embodiments in which sensor modules and processing modules are enclosed within a single device and the single device includes sensors, the processing unit may need to directly or indirectly be in contact with a body of a human or animal.

In some embodiment, processing units, sensor modules, or a combination thereof may be individually or collectively self-powered through a battery, capacitor bank, or other means of electric energy storage. In some further embodiment, the means of electric energy storage may be rechargeable by wired or wireless means.

According to some example embodiments of a system for predicting an onset of a neurological event in a human or animal, a processing device includes a communication module. The communication module may enable wired or wireless communication with a sensing module or therapeutic module through optical, electromagnetic, acoustic, or other means as described herein. Electromagnetic means may include but are not limited to Bluetooth, Wi-Fi, MiCS, cellular networks, satellite communication, microwave communication, and radio broadcast. Such electromagnetic means may also be used to connect the processing unit to the internet for remote monitoring, alarms, offsite processing, and digital records among others. In some embodiment the processing device may connect to a smart phone through the internet and monitor system function and execute alarms through a phone-based application.

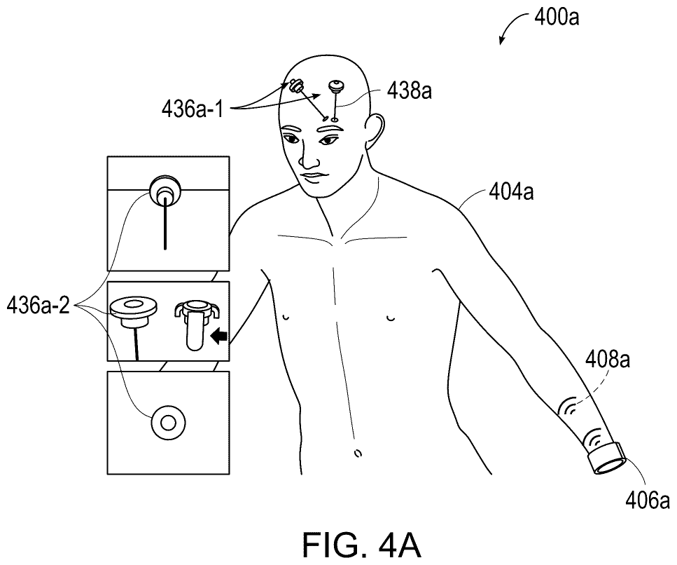

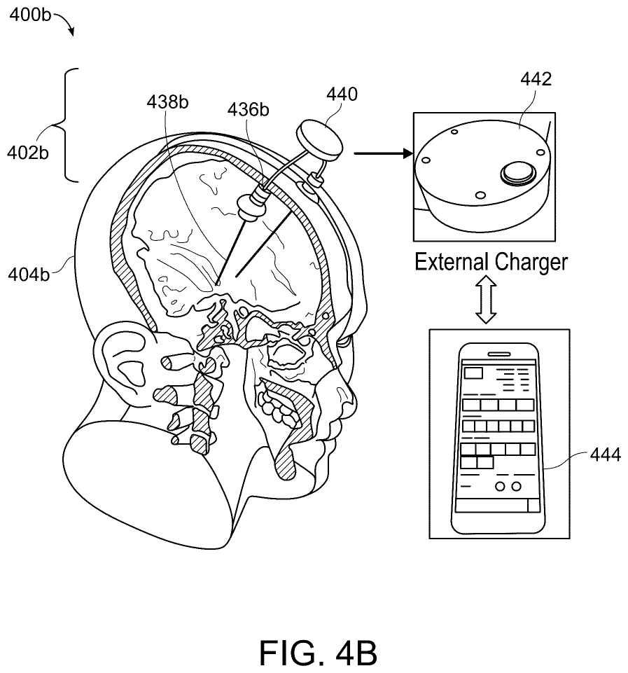

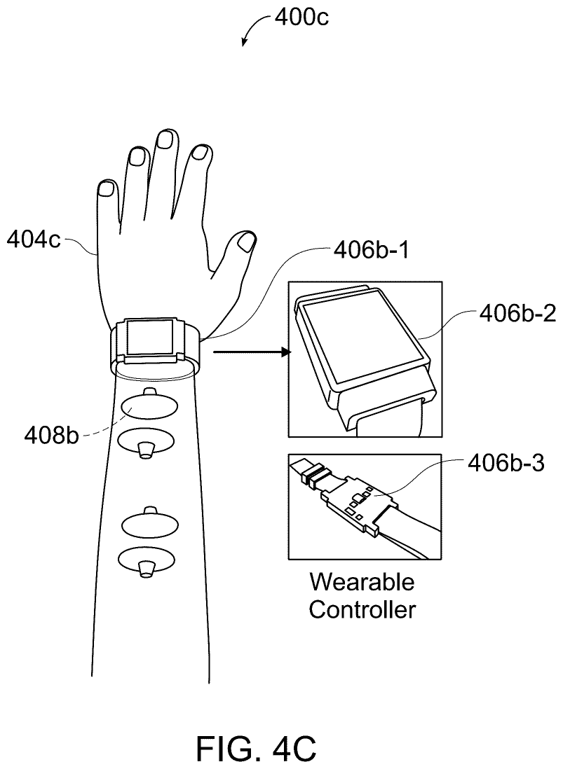

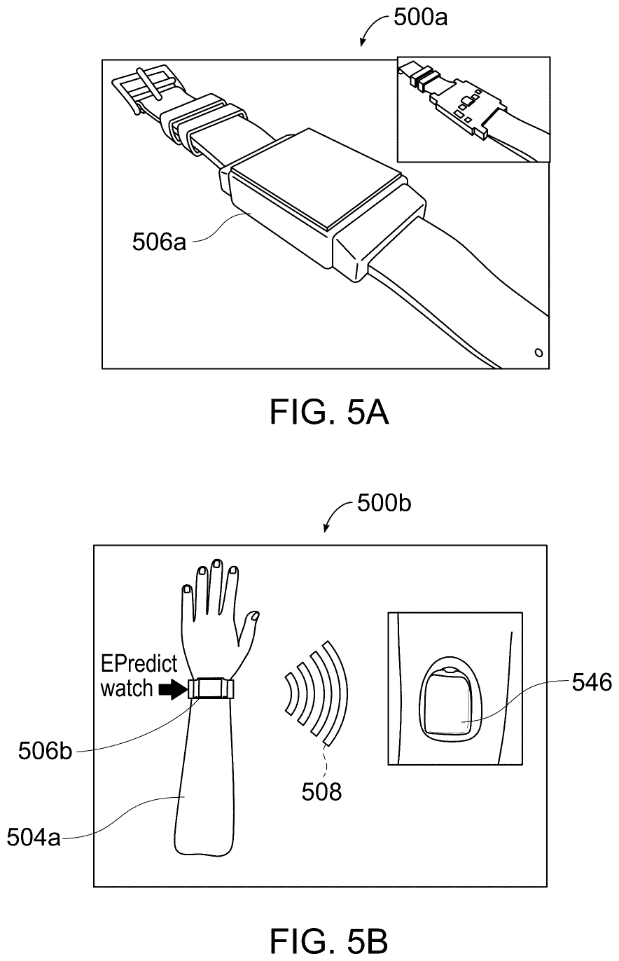

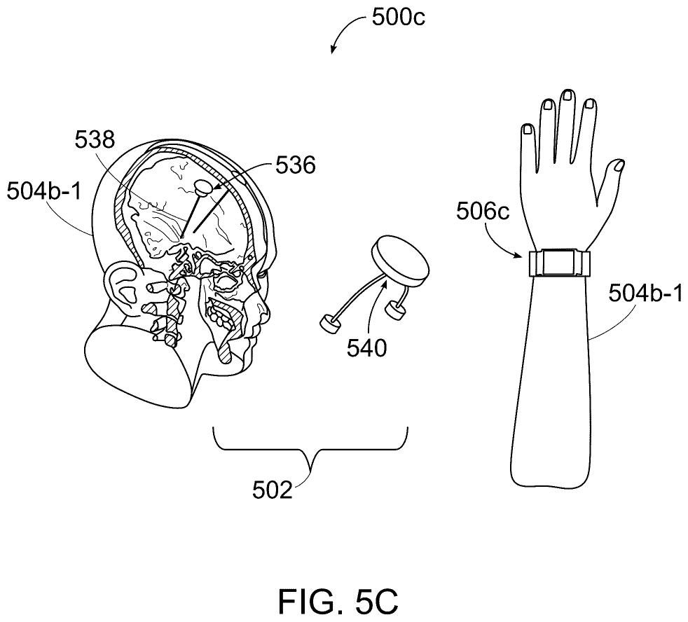

FIGS. 4A-5C generally illustrate example embodiments of systems and devices for predicting an onset of a neurological event in a human or animal.

FIG. 4A illustrates an example embodiment of a portable system 400a for predicting an onset of a neurological event in a human 404a or animal including sensing modules, e.g., 436a-1, configured to measure an electroencephalography (EEG) signal or for delivering a therapeutic stimulation, with inset images showing an example sensing module 436a-2. The portable system 400a may be system to the portable system 100 and similar elements are indicated by similar reference numbers but incremented by 300. The portable system 400a includes a processing module 406a configured to be wearable on the human 404a and communicatively coupled with the sensing modules 436a-1 using a communication path 408a, which may be a wireless communication path. The sensing module 436a-1, including the example sensing module 436a-2, may include wireless stimulation leads 438a, the stimulation leads further configured to be implantable within a skull of the human 404a, for example, through small incisions in the skull The stimulation leads 438a may be configured for sensing, e.g., measuring electroencephalography signals, and for delivering a therapeutic intervention, e.g., a deep brain stimulation.

FIG. 4B illustrates a sensing module 402b of a system 400b similar to the portable system 400a of FIG. 4A, with insets showing an external charger 442 and a handheld device 444 to which the portable system is communicatively coupled. The sensing module 402b includes sensing elements, e.g., 436b, with implantable leads, e.g., 438b, implanted in a skull of a human 404b. The sensing module 402b may further include an external gateway 440, which may be placed external to the skull of the human 404b, communicatively coupled to the sensing elements, e.g., 436b, and configured to transmit physiological signals acquired by the sensing elements, e.g., 436b, to a processing module. The sensing module 402b may further include the external charger 442 for charging the external gateway. The external gateway 440, the external charger 442, or a combination thereof may be communicatively coupled to a handheld device 444. The handheld device 444 may include the processing module or may also be communicatively coupled to the processing module. The handheld device 444 may also be configured to enable delivery of a therapeutic intervention through the implantable leads, e.g., 438b.

FIG. 4C illustrates a processing module 406b-1 of a system 400c similar to the system 400a of FIG. 4A, with insets showing example wearable embodiments of processing modules 406b-2, 406b-3. The processing module 406b-1 may be a wearable device, for example, a smartwatch or a wrist-worn device on a human 404c. The processing module 406b-1 may be communicatively coupled to a sensing module through a wireless communication path 408b.

FIG. 5A illustrates an example embodiment of a processing module 506a of a portable system 500a for predicting an onset of a neurological event in a human or animal. The processing module 506a may be similar to the processing module 406b-1 described herein with reference to FIG. 4C and may be configured to be worn on a wrist of the human.

FIG. 5B schematically illustrates an example embodiment of a portable system 500b for predicting an onset of a neurological event in a human 504a or animal including a processing module 506b communicatively coupled to a therapeutic module 546 configured to deliver therapeutic intervention for the neurological event. The processing module 506b, which may be a portable or wearable device, e.g., a watch, is communicatively coupled to the therapeutic module 546 through a wireless communication path 508. Examples of the therapeutic device 546 as illustrated in FIG. 5B may include a drug pump.

FIG. 5C schematically illustrates an example embodiment of a portable system 500c for predicting an onset of a neurological event in a human 504b-1, 504b-2 or animal, the system including a sensing module 502 with implantable electrical leads for measuring electroencephalography signals and a wearable processing module 506c. The sensing module 502 includes an external gateway 540 configured to communicatively couple with sensing elements, e.g., 536, and the implantable electrical leads, e.g., 538. The sensing module 502 is communicatively coupled to the processing module 506c, which may be configured to perform the predicting of the onset of the neurological event onboard, locally or proximally to the human 504b-1, 504b-2.

### Signal Processing, Artificial Intelligence Training and Execution, and Deep Learning Signal Preconditioning

For embodiments associated with epileptic seizure prediction, a system for predicting an onset of neurological events, i.e., seizures, may ideally predict such an event from hours in advance to a minute in advance. Clinical input suggests that predictions from 1 hour to 5 minutes in advance may result in better patient outcomes. To enable prediction of an impending seizure, the system may need to classify or differentiate between pre-seizure and non-seizure states within data samples representative of physiological signals. Because most of the time patients are not in a pre-seizure state (seizure for many patients are not frequent or daily events), existing signal datasets of physiological measurements in epilepsy patients may be imbalanced, emphasizing a rarity the pre-seizure states compared to non-seizure states.

In some example embodiments of a system configured to acquire EEG, ECG, or combined EE and ECG signals, pre-processing may be utilized. In some further example embodiments, the pre-processing utilized may include techniques or protocols that simple and relatively impose low demand on power consumption; as a result, such embodiments may not include higher order processes. Filtering techniques to be implemented in the system may include but are not limited to low-pass, high-pass, band-pass and band-stop, Laplace, Fourier, Finite Impulse Response (FIR), Infinite Impulse Response (IIR), Butterworth, notch filter, power-line noise, band pass, Chebyshev, Elliptic filters, analogue, hardware based, active, passive, linear, and non-linear filters, among others. However, pre-processing filters, including notch-filters for power-line noise and band pass filters, may not help improve results significantly.

According to some embodiments, deep learning algorithms may also be used to pre-process a signal before using it for neurological event characterization, prediction, or identification. Pre-processing may be accomplished in a sensor device using limited processing hardware in order to improve or minimize an amount of data being communicated between devices or modules. AI algorithms may also be used during pre-processing so as to identify and transmit only signal markers or characteristics which may be used in the AI model for predictive, characterization or identification purposes by a processing device.

### AI Algorithms and Deep Learning models

Embodiments of AI software to be used in the processing device may include algorithms based on supervised learning, unsupervised learning, reinforcement learning, natural language processing, evolutionary algorithms, ensemble algorithms, and deep learning algorithms. In some embodiments, deep learning algorithms are used. Types of deep learning algorithms that may be used, which may be selected based upon application or pathophysiology, include Convolutional Neural Networks (CNNs), Recurrent Neural Networks (RNNs), Long Short-Term Memory (LSTM) networks, Generative Adversarial Networks (GANs), Autoencoders, Deep Belief Networks (DBNs), and Multilayer Perceptron's (MLPs). The AI software may be implemented in a computer-implemented method.

For some embodiments described herein, including a system for prediction of an onset of a neurological event of a human or animal and wherein the neurological event is a seizure in an epilepsy patient, CNNs may be used in a processing unit (or processing module or device) including an FPGA configured to execute an algorithm based on the CNNs. In other to reduce overall processing power required for characterizing, predicting, and identifying neurological events, a pruned CNN may be used to remove weights that may not be used or may be of lower impact upon the characterizing, prediction or identifying to provide a more lightweight algorithm or to reduce processing requirements.

In some embodiments, the processing unit may be configured to execute a DL model for seizure prediction based on ECG signals, EEG signals, iEEG signals or a combination thereof.

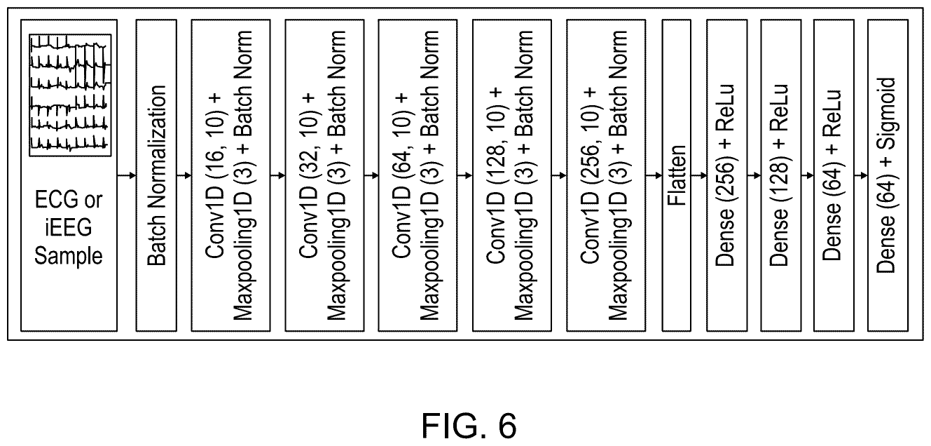

FIG. 6 illustrates an architecture of a convolutional neural network that may be implemented by an example embodiment of a system for predicting an onset of a neurological event in a human or animal. The architecture of the CNN of FIG. 6 may be an example of a particular embodiment. Raw ECG, raw iEEG, or raw EEG samples may undergo batch normalization to enhance their suitability for subsequent processing. The batch normalized samples may be passed through a series of layers. Each batch normalized sample of the batch normalized samples may be passed through a series of five 1-dimensional CNN blocks, each 1-dimensional CNN block accompanied by a Max-pooling layer. The 1-dimensional CNN blocks may be configured to extract latent features with maximum information content. Utilizing 1-D convolutional layers may enable a DL model to capture crucial features from each sample effectively. The features extracted by the CNN blocks may be flattened and passed through four dense layers for binary classification. While choices of activation function for the four dense layers may vary, a specific embodiment may include a rectified linear unit (ReLU) activation function for the first three dense layers of the four dense layers and a Sigmoid activation function for the final layer. This particular embodiment may be optimized to balance computational efficiency and accuracy.

CNN models, such as the model described herein as a particular embodiment with reference to FIG. 6, may be suited toward capturing spatial patterns within at least one of iEEG, EEG, or ECG signals and extracting nuance features, hence identifying intricate seizure-related patterns more effectively. The CNN architecture shown in FIG. 2 remains consistent across all patients for initial training; however, individualized training (personalized training) may also be used in some embodiments. Embodiments including personalized training may be based on creating distinct trained models specific to each patient. The distinct, patient specific trained models may be obtained by refining a common model with patient-specific data.

### Training of Neural Network

In some embodiments, where data is balanced for CNN training, additional use of balancing functions may not be required. Examples of such embodiments may include systems in which a neurological event for which an onset is predicted is a commonly or frequently occurring neurological event and, thereby, a presence of neurological events in training data may be balanced with periods of neurological function. Examples of such neurological events may include migraines, consistent migraines, tremors, or readily occurring seizures. However, for many patients, biomedical neurological data may be imbalanced as neurological events do not occur often, i.e., a majority of signals or data acquired from such a patient is associated with normal function.

In some embodiments of a system utilizing ECG, EEG, or iEEG data or a combination thereof for epileptic seizure prediction, training an AI model for predicting an onset of a neurological event, i.e., a seizure, may require additional processing due to unbalanced training data as seizures may be rare in occurrence compared to periods without neurological events. Training neural networks for biomedical tasks can be challenging, at least in part due to uneven and inconsistent distribution of labels (e.g., pre-seizure or non-seizure). Balancing such a training set while retaining the valuable information contained therein may provide optimal training results for embodiments of the system described herein. A particular approach in some embodiments for addressing the imbalance of training data may include using a loss function, specifically, a Focal Loss function, which may address issues of class imbalance more effectively than a balanced cross-entropy (BCE) loss function. The focal loss function may be defined as:

$$FL ( {p}_{t} ) = \{ \begin{matrix}- {α ( 1 - p )}^{γ} log p , & y = 1 \\ - ( 1 - α ) {p}^{γ} log ( 1 - p ) , & otherwise\end{matrix}$$

where, p∈[0,1] represents a model's estimated probability for each class and y is an actual label of a given class. We consider y=1 for pre-seizure periods and y=0 for non-seizures.

The Focal Loss function as described herein may be tuned via two parameters: α, which may be used similarly to an imbalanced BCE loss function by assigning predefined weights to different classes, and γ, which may improve cross-entropy behavior by assigning a lower loss to misclassified samples. For a specific embodiment, an exhaustive search may be performed to determine exemplary parameter values of α=0.2 and γ=2 to yield optimal performance across all patients.

Such a weighting strategy may help ensure that the model does not favor non-seizure instances and may effectively combat biases of unbalanced datasets to improve the overall classification performance.

### Time and Channel Voting

Use of a single time point for forming a prediction of an onset of a neurological event may be susceptible to noise or other confounding events or features. In some embodiments, multiple data samples, for example, data samples spanning multiple time points or multiple input channels, may be used collectively to improve a predictive power of a trained AI model.

In some embodiments, a processing system includes voting schemes to improve performance and mitigate risk of false detection based on a single faulty sample. In a specific example of a particular embodiment, a majority voting strategy may be employed for both channels and time. For single-channel signals (single-channel ECG, EEG, temperature, mechanical, optical, or other single-channel sensor signals), time voting may include storing decisions from a number of most recent samples into a memory buffer (in the particular embodiment, at least 15 samples over 60 seconds of recording) and determining a final decision based on the majority vote. The decisions may be generated by a trained AI model and may be used to identify, characterize, and predict a neurological event (in the particular embodiment, an epileptic seizure) for a specific time sample or number of samples (time-proving or sample-proving period). The decisions may be voted on, implying that a predominant decision for the selected period or number of samples will be the final decision.

An example voting scheme may be implemented in a computer-implemented method that includes forming decisions of an onset of a neurological event within 15 minutes following forming of the prediction. Forming the prediction may include recording samples over a period of one minute (time-proving period of one minute). If decisions are made every 2 seconds, a total of 30 decisions (sample-proving period of 30) occur during the minute associated with the prediction of whether a seizure will occur in the next 15 minutes. Therefore, if a majority of the decisions indicate that a seizure will occur in the next 15 minutes, the final predictive decision is positive for a seizure, despite some negative predictions, which are a minority of a total number of the total decisions predicted, calculated during that 1 minute proving period. In most embodiments, there may be no limits on how large or small the proving periods can be. In some embodiments, to predict an onset of a seizure from greater than 0 seconds to 1 hour or more following forming of a prediction, proving periods may range between 30 seconds and 15 minutes, although other combinations are allowed.

In some embodiments, the prediction may identify a risk period, i.e., a period during which a neurological event may occur, and the risk period may span a period of time following a time of prediction. AI models may be trained to identify onsets of neurological events within risk periods of a specific time window or length.

In some example embodiments including multichannel signals (multi-channel ECG, multichannel iEEG signals, and other types of multichannel signals from the body), decisions may be collected for all channels, and a majority vote approach, as described above, may be implemented to reach a final decision. Restated, decisions based on data from each single-channel may be pooled, and a final decision reflects a majority of the decisions based on single-channel data. For example, if a possible onset of a seizure within a known time period is pooled, and the majority of decisions indicate that a seizure will not occur within that time, then the final overall decision is negative for seizure onset. In some embodiments, voting schemes for both channel and time voting may be utilized. In such embodiments, decisions from one or multiple channels may be buffered for a proving period and voted on to achieve a majority decision for each channel, and then a majority vote for all channels is executed for a final combined decision.

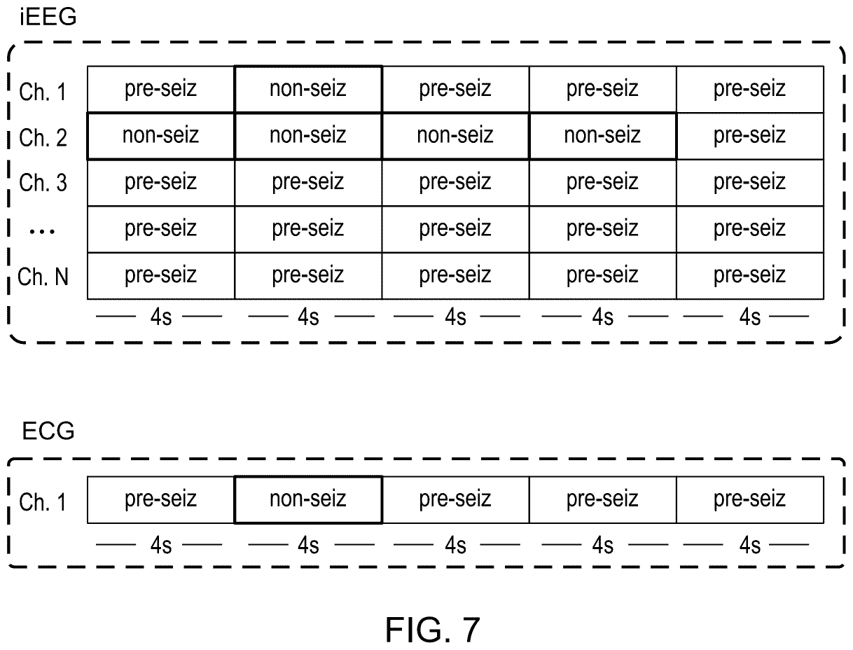

FIG. 7 illustrates a voting process that may be implemented in an example embodiment of a portable system as a computer-implemented method for predicting an onset of a neurological event in a human or animal, the system configured to acquire intracranial electroencephalography and electrocardiography signals. The voting scheme of FIG. 7 indicates a single ECG channel and N iEEG channels. A physiological signal comprising five time intervals, each of 4 seconds, is acquired for each channel. The voting scheme may determine a majority vote for each channel and may subsequently use the majority vote for each channel to form a prediction of the onset of the neurological event, e.g., a subsequent majority vote. Alternatively, the voting scheme may determine a majority vote for all time intervals across all channels to form the prediction.

In some embodiments, a sensing module, e.g., the sensing module 102-1, 102-2 described herein with reference to FIG. 1A, or a processing module, e.g., the processing module 106, may be configured to partition a given physiological signal into intervals. For a given interval of the intervals, the processing module may be configured to employ one or more metrics of the given interval to calculate a respective likelihood of an onset of a neurological event. Identifying a risk period of the onset of the neurological event may include executing a voting scheme, for example, the voting scheme described herein with reference to FIG. 7, based on the respective intervals determined. In other embodiments including a plurality of physiological signals, for example, the system 100 described herein with reference to FIG. 1A, a voting scheme may include voting across the plurality of physiological signals. In some embodiments, a voting scheme may be implemented across channels of physiological signals and across partitioned intervals for each channel.

Alternatives to voting schemes as described herein may also be implemented to obtain a final decision based on further processing of discrete decisions for a given interval or channel. In some embodiments, other forms of mathematical or statistical processing may or may not be included, for example, to determine the final prediction. For example, decisions in time or per channel may be converted to numerical values and averaged. The final decision may be obtained if the average (or a weighted average) exceeds a specified static or dynamic threshold, or any other types of thresholds, limits, or tendences. In some embodiments, other calculations for variable grouping multiple decisions or central tendencies (besides averaging) may be used, including but not limited to mean, median, mode, distributions, tendences, differences, variability, or deviations. In some embodiments, statistical means may for determining a most likely overall decision may be implemented for both a qualitative description of a discrete decision in time per channel or quantitative variables. For example, mathematical formulations for normal or non-normal distributed data may be implemented. Statistical approaches for final decision making may also include assumptions, parametric non-parametric tests, fit tests, regressions, data type tests, data distribution tests, scaling tests. The tests described herein may further include univariate or multivariate tests, number of samples, statistical estimates, outliers or reliability thresholds, Anova and all its derivative tests. Further mathematical processing may include analysis of numerical dynamics, rates of change analysis, and other non-static analysis of decision variables

### Example Configurations for ECG and or ECG-Based Systems for Seizure Prediction

As described herein, embodiments of a system for predicting an onset of a neurological event in a human or animal may employ a deep learning algorithm for efficient, accurate, energy-efficient, in situ (locally placed on or near a body of the human or animal, without a need for internet connectivity) prediction of the neurological event, e.g., epileptic seizures. In some example embodiments, the system may utilize a combination of features described herein, for example, processing modules or sensing modules using miniaturized and limited computational hardware to perform predictive analyses, which may enable wearable or implantable components for example systems, as well as multi-channel sensing and voting schemes for improved prediction forming. DL algorithms include CNN or CNN-based networks and, in some embodiments, pruned CNN algorithms. The system in such example embodiments may use limited signals to achieve predictions.

In an example of an embodiment, a system utilizes only ECG signals to predict seizures. The predictive system may be integrated into a wearable device to inform a patient, caregivers of the patient, or others of a possible onset of a seizure. Such a system may also inform a treatment device/system to control therapy through a feedback loop as shown in FIG. 5. In the example of the embodiment, two wearable devices are used, a processing unit and a sensing unit. The sensing unit may contain an ECG sensor module and a sensor communication module. The processing unit may be configured to execute a DL algorithm on data acquired by the sensing unit.

The ECG sensor module acquires electrical ECG signals from a heart the patient through skin using electrodes. The sensing module may also filter or process a given ECG signal prior to transmitting it to the processing device via the sensor communication module. In the example, the sensor communication module wirelessly transmits, for example, using a Bluetooth communication protocol, the signal from the sensing device to the processing device. Furthermore, in this example embodiment, the sensing unit includes a case that houses at least a portion of electronics of the sensing unit. This case is releasably connected to an elastomeric (silicone, polyurethane) patch with electrodes and the patch may be releasably adhered to the skin of a patient. The case containing the electronics may be detached as needed throughout the day, while the elastomeric patch may be replaced as required.

In the example of the embodiment, the processing unit is a device that may be worn on the body or a device that may be carried by the patient during daily activities, for example, a pocket-sized device to be carried within the patient's clothing.

The processing unit includes at least a processor communication module communicatively coupled to the sensing module, the processing unit receiving data from the sensing device, and a processing module that implements DL software. The processor communication module of the processing unit may also connect to the internet as required. The processing module uses miniaturized programable software and hardware architectures, for example, FPGAs, ASICs, or a combination thereof, to execute the DL software.

In the example of the embodiment, the deep learning software implemented in the processing unit is a pruned CNN; the DL software may pre-process (e.g., resampling, normalizing, cropping, or other pre-processing techniques) a signal acquired by the sensing unit to improve compatibility with DL algorithms. Individual decisions, or likelihoods calculated, generated for at least a portion of samples of the signal, of a possible occurrence of a seizure as generated from the DL algorithms are further utilized in a voting scheme to improve accuracy of a final prediction. In the example embodiment, the voting scheme is associated with individual decisions or individual metrics determined for time-based sampling and, in systems with multiple sensing channels, the voting scheme may further be associated with at least a portion of the multiple sensing channels.

In an example variation of the example embodiment, the DL software is trained using a repository including generalized patient data to achieve an initial level of accuracy for the system, which is communicated to the patient. The DL algorithms are further trained using patient-specific data (from the same patient) during an initial personalized training period to enhance prediction accuracy. After personalized training, an updated DL algorithm may be implemented in the system to predict in advance the onset of a seizure and inform the patient caregivers of the patient, or a feedback-loop therapeutic treatment system. Further training (or refinement) of the DL algorithms may be performed locally, that is, on a module such as the processing module associated with the system, or on a separate device, wherein the refined DL algorithms are transferred to the system for implementation.

### Example Combined Signal Embodiments

In example validated embodiments, signals acquired using intracranial EEG, extracranial EEG, or ECG signals from epilepsy patients are used to train personalized prediction algorithms. Data may be down-sampled, processed using a focal-loss-function to address inherent imbalances of a data set, and used to train CNNs. Processing modules of an example validated system may implement the CNNs trained and further include a voting scheme for enhancing sensitivity and specificity of predictions formed by the processing modules. The processing module further accepts multi-modal input signals, e.g., EEG (iEEG or external EEG) and ECG, acquired using communicatively coupled sensing modules and utilizes at least a portion of the multi-modal input signals in forming the predictions to enhance accuracy of the predictions formed and minimize false alarms. Computational efficiency may be optimized to implement the CNNs trained and voting schemes in a miniaturized, energy efficient FPGA. The CNNs trained may be further refined using patient-specific data following training.

FIG. 8 illustrates a plot of average sensitivity, specificity, and accuracy of epileptic seizure prediction across a cohort of subjects according to an example embodiment. A CNN-based algorithm utilizing the validated embodiments described herein may achieve an accuracy, sensitivity, and specificity greater than 99% in predicting a seizure 1 hr before onset of the seizure using iEEG or ECG alone. Embodiments that use a combined, multi-modal iEEG and ECG approach, in combination with the voting scheme mechanism result in high specificity, sensitivity, and accuracy as well. Useful features of embodiments of the AI models and systems described herein may include high specificity (very low false positive rates) and low computational cost (lightweight CNN models without needing additional feature extraction, which may make the embodiments more practical and feasible for implementation on resource-restricted devices.

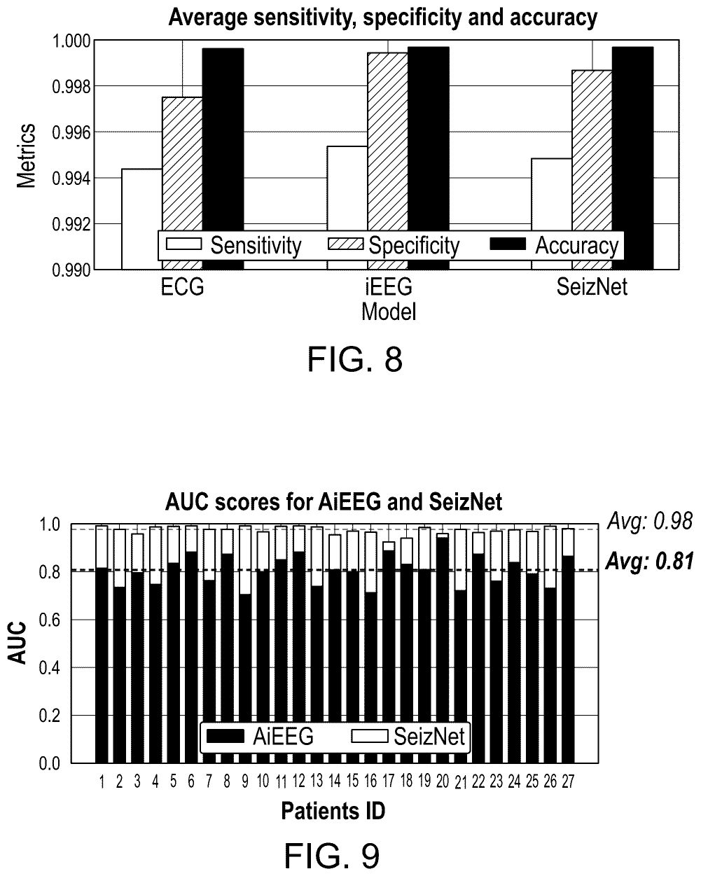

FIG. 9 illustrates a plot comparing area under the curve (AUC) metrics for an example embodiment of the present invention and a previously reported epileptic seizure prediction technique. For a given patient of a set of patients, physiological signals of the other patients of the set of patients are used to train a model for predicting the onset of a seizure. The model may further be refined by patient-specific data for the given patient. The present invention (SeizNet) indicates an average 17% improvement in prediction accuracy over the previously reported technique (AiEEG).

### Other Example Applications

Seizure prediction and closed-loop preventive mechanisms that can stimulate using a Deep Brain Stimulation (DBS) system may be useful for patients who are unresponsive to medication, known as drug-resistant or refractory epilepsy. As described herein with reference to FIG. 4B, implanted leads monitor iEEG signals, while a portable or wearable device (such as a watch, armband, or pocket device) predicts seizures based on EEG signals. When a prediction is positive, the wearable may send a signal to the DBS system to initiate preemptive stimulation through the leads to prevent or mitigate the seizure.

Example embodiments of the present invention as described herein may also be adapted and used for other types of disease, e.g., Parkinson's Disease. An example embodiment of portable system of the present invention for PD may include an FPGA in a processing device containing modules for a Deep Learning Network (DLN) and a physical communication layer. Al algorithms in the FPGA may classify tremors into pre-determined thresholds/groups, select clinical-based stimulation characteristics (amplitude, pulse duration, frequency, and what is the optimal electrode to execute the stimulations), and transmit to the stimulation characteristics selected to a stimulation lead for execution of new stimulation settings.

In example embodiments of the present invention for PD, an accelerometer and or a gyroscope closed-loop system may be implemented on a wearable (or implantable) device to maintain a small form factor. The closed loop system for PD may use DL algorithms specifically designed to characterize tremors. In some embodiments, computationally efficient deep learning algorithms may be used. While larger DLNs may typically learn more abstract features and offer improve performance, the larger DLNs may not be implementable within a small form factor FPGA without sacrificing performance. Alternatives to implementing the larger DLNs may include removing DLN weights that have little or no effect on the network or quantizing weight by reducing a number of bits that represent a network parameter. Quantizing weights, however, may alter DLN output values. DLN model optimization techniques that convert a trained network to a common Intermediate Representation (IR) using various training frameworks and clean the DLN for inference may be used for embodiments with larger DLNs. Several components used during training that slow down calculations (batching operators, fuse layers) may be removed using this approach. To date this approach has been successful in tremor recognition using a miniaturized FPGA.

In example embodiments of the present invention for tremor identification and characterization, Convolutional Neural Network designs may use accelerometer and gyroscope (AG) data, which may be low-pass filtered to reduce noise and isolate desired frequency components. Lightweight feature extraction may be used for differentiation.

In example embodiments of systems for predicting tremors in PD patients, AI-based recognition and classification for tremors and stimulation strategies may use a Gated Recurrent Unit (GRU) architecture as a robust and efficient approach to process data from the accelerometer, the gyroscope, and any additional sensors, and classify the tremor. In further embodiments of the systems for predicting tremors in PD patients, dyskinesia and bradykinesia may be better treated by target selection (as opposed to electrode selection) for a multi-lead stimulation device. In further embodiments, DL or AI algorithms may be trained and personalized. Part of the personalization of the AI algorithm may include identifying stimulation parameters that may lead to improved tremor outcomes.

In additional example embodiments of systems for predicting an onset of a neurological event, data from ECG signals, EEG signals, other types of biological sensor data (temperature, mechanic, optical), or a combination thereof may be used to predict the onset of a migraine in advance. In some embodiments, a CNN algorithm implemented and trained in a processing module located in a portable device may be used to predict a migraine from ECG or EEG signals. This may allow for the patient to pre-medicate themselves to stop or mitigate the migraine before it occurs.

In other example embodiments of systems for predicting an onset of a neurological event, a smart predictive device that uses artificial intelligence to anticipate a seizure, a migraine, a tremor, or other neurological events may be used in conjunction with a medication infusion pump, similar to those used for diabetes (insulin infusion pumps), in order to preemptively treat an event through a preemptive injection of medication into the patient. The drug infusion pump may be similar to the therapeutic module 546 described herein with reference to FIG. 5B. In such embodiments, signals from sensors (for examples, standalone ECG, standalone EEG, or a combination of ECG and EEG) may be employed to predict the onset of a seizure using AI in a portable processing unit. In such events, the processing unit may wirelessly transmit prediction information to the infusion pump, which may then inject a predetermined or calculated volume of medication into the patient. For epileptic seizures, the pump may inject fast-acting anti-epileptic drugs, or, for migraines, anti-pain medication may be injected. A potential advantage of such predictive systems may include that a needle of the infusion pump may not need to penetrate skin of a patient until a predicted time is established, only penetrating when a need for delivering medication is identified. Prior to the forming of a prediction of an impending neurological event, the needle may remain retracted, which may reduce pain or discomfort for the patient and a risk of infection. It is understood that a closed-loop system for medication infusion may be required for some patients, while for others, merely predicting the onset of a seizure or migraine may enable pre-medicating through other means such as oral medication, injection, aspiration, microneedle patches, drops, among others.

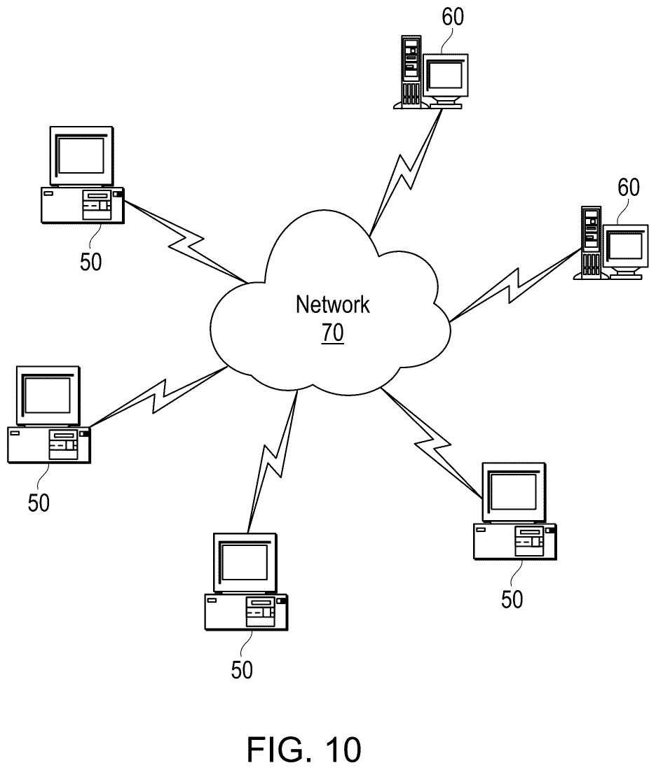

FIG. 10 illustrates a computer network or similar digital processing environment in which embodiments of the present disclosure may be implemented.

Client computer(s)/device(s) 50 and server computer(s) 60 provide processing, storage, and input/output devices executing application programs and the like. The client computer(s)/devices 50 can also be linked through communications network 70 to other computing devices, including other client computer(s)/device(s) 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

Client computer(s)/device(s) 50 and/or server computer(s) 60 may be configured, alone or in combination, to implement the embodiments described herein, e.g., the method 1900, among other examples. The server computer(s) 60 may not be separate server computers but part of communications network 70.

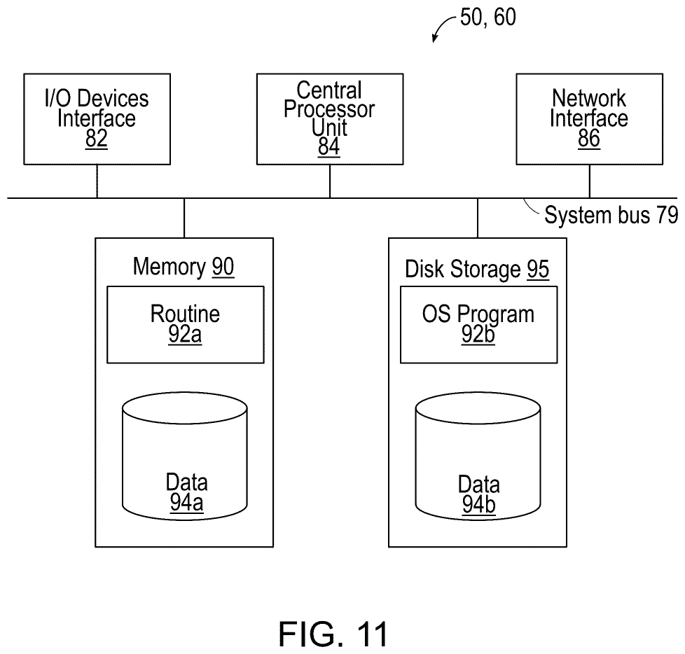

FIG. 11 is a diagram of an example internal structure of a computer (e.g., client computer(s)/device(s) 50 or server computer(s) 60) in the computer system of FIG. 10. Each computer/device 50 and server computer 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output (I/O) ports, network ports, etc.) and enables the transfer of information between the elements. Attached to the system bus 79 is an I/O devices interface 82 for connecting various input and output devices (e.g., keyboard, mouse, displays, printers, speakers, etc.) to the computer/device 50 or server computer 60. A network interface 86 allows the computer/device 50 or server computer 60 to connect to various other devices attached to a network (e.g., communications network 70 of FIG. 20). Memory 90 provides volatile storage for computer software instructions 92a and data 94a used to implement an embodiment of the present disclosure (e.g., the method 1900, among others). Disk storage 95 provides non-volatile storage for computer software instructions 92b and data 94b used to implement an embodiment of the present disclosure. A central processor unit 84 is also attached to the system bus 79 and provides for the execution of computer instructions.

Embodiments or aspects thereof may be implemented in the form of hardware including but not limited to hardware circuitry, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A portable system for predicting an onset of a neurological event in a human or animal, the portable system comprising:
one or more sensing modules configured to acquire one or more physiological signals from the human or animal;
a processing module, communicatively coupled to the one or more sensing modules, configured to analyze the one or more physiological signals acquired, the analyzing including:
employing one or more metrics of the one or more physiological signals to calculate a likelihood of the onset of the neurological event; and
identifying a risk period of the onset of the neurological event based on the likelihood calculated.

2. The portable system of claim 1, wherein the processing module is configured to calculate the likelihood of the onset of the neurological event by executing a machine learning algorithm.

3. The portable system of claim 2, wherein the machine learning algorithm includes a supervised learning, unsupervised learning, reinforcement learning, natural language processing, evolutionary, ensemble, or deep learning algorithm.

4. The portable system of claim 3, wherein the deep learning algorithm includes a convolutional neural network, pruned convolutional neural network, recurrent neural network, long short-term memory network, generative adversarial network, autoencoder, deep belief network, or multilayer perceptron.

5. The portable system of claim 2, wherein the processing module includes a training module configured to perform at least one of training the machine learning algorithm using a repository comprising data of a similar type to the one or more physiological signals or refining a pre-trained machine learning algorithm using patient-specific data of a similar type to the one or more physiological signals.

6. The portable system of claim 1, wherein the one or more sensing modules or the processing module is further configured to partition a signal of the one or more physiological signals into a plurality of intervals, and wherein the processing module is further configured to employ one or more metrics of an interval of the plurality of intervals to calculate a respective likelihood of the onset of the neurological event and to identify the risk period based on the respective likelihoods calculated.

7. The portable system of claim 1, wherein the likelihood calculated is a first likelihood and wherein the processing module is configured to employ the one or more metrics of the one or more physiological signals to calculate at least one additional likelihood.

8. The portable system of claim 7, wherein the processing module includes a voting module configured to form the prediction based on the likelihood and the at least one additional likelihood calculated.

9. The portable system of claim 1, wherein the one or more sensing modules are configured to acquire an electrocardiography, electroencephalography, temperature, heart rate, accelerometry, electromyography, or electrodermal signal.

10. The portable system of claim 1, wherein the one or more sensing modules acquire physiological signals using at least two physiological measurement modalities.

11. The portable system of claim 1, wherein the sensing module and the processing module are configured to communicate via a communications path including at least one link employing a wireless communication protocol.

12. The portable system of claim 11, wherein the wireless communication protocol is a Bluetooth-based communication protocol or an ultrasound-based communication protocol.

13. The portable system of claim 1, further comprising a therapeutic module communicatively coupled to the processing module, the therapeutic module configured to apply a therapeutic intervention based on the prediction of the onset of the neurological event.

14. The portable system of claim 13, wherein the therapeutic module includes a drug infusion pump or a neurostimulation device.

15. The portable system of claim 1, wherein the neurological event is an epileptic seizure, a tremor, or a migraine.

16. The portable system of claim 1, wherein the processing module is configured to analyze the one or more physiological signals within a proximity of short-range wireless communication from the sensing module.

17. A method for predicting an onset of a neurological event in a human or animal, the method comprising:
acquiring one or more physiological signals of the human or animal;
analyzing the one or more physiological signals acquired, including:
employing one or more metrics of the one or more physiological signals to calculate a likelihood of the onset of the neurological event; and
identifying a risk period of the onset of the neurological event based on the likelihood calculated.

18. The method of claim 17, wherein calculating the likelihood of the onset of the neurological event includes executing a machine learning algorithm for the one or more physiological signals.

19. The method of claim 18, further comprising training the machine learning algorithm based on a repository including data of a similar type to the one or more physiological signals, still further comprising refining the machine learning algorithm trained using the one or more physiological signals acquired of the human or animal.

20. The method of claim 17, further comprising delivering a therapeutic intervention to the human or animal based on the prediction formed.

21. The method of claim 17, further comprising partitioning a signal of the one or more physiological signals into a plurality of intervals, and wherein analyzing the one or more physiological signals includes employing one or more metrics of an interval of the plurality of intervals calculate a respective likelihood of the onset and identifying the risk period based on the respective likelihoods calculated.

22. The method of claim 17, wherein calculating the likelihood of the onset of the neurological event includes calculating at least two likelihoods, and wherein identifying the risk period based on the likelihood calculated further includes performing a voting scheme on the at least two likelihoods.

23. A method for predicting an onset of a neurological event in a human or animal, the method comprising:
employing one or more metrics of at least one physiological signal acquired from the human or animal, to calculate a likelihood of the onset of the neurological event;
identifying a risk period of the onset of the neurological event based on the likelihood calculated; and
forwarding a representation of the risk period identified to the human or animal, to a caregiver of the human or animal, or to a therapeutic module arranged to apply a therapy to the human or animal.
